(19) United States
(12) Patent Application Publication
(10) Pub. No.: US 2017/0273773 A1
(43) Pub. Date: Sep. 28, 2017

(54) MEDICAL DEVICE FOR HEART DISEASE

(71) Applicant: TERUMO KABUSHIKI KAISHA, Tokyo (JP)

(72) Inventor: Kouichirou YORI, Ashigarakami-gun (JP)

(73) Assignee: TERUMO KABUSHIKI KAISHA, Tokyo (JP)

(21) Appl. No.: 15/469,765

(22) Filed: Mar. 27, 2017

(30) Foreign Application Priority Data
Mar. 28, 2016 (JP) 2016-063396

(51) Int. Cl. A61F 2/02; A61F 2/24

(52) U.S. Cl. CPC A61F 2/02; A61F 2/2481; A61F 2/24; A61F 2/2478; A61F 2220/0016

(57) ABSTRACT

A medical device is disclosed, which is capable of treating cardiac insufficiency, on a causal therapy basis. The medical device is a heart harness composed of a porous hollow structure. A method of treating a heart disease includes applying the medical device as above to a heart of a subject in need thereof.

### CROSS-REFERENCES TO RELATED APPLICATIONS

The present application claims priority to Japanese Patent Application No. 2016-063396 filed on Mar. 28, 2016, entitled “Medical Device for Heart Disease,” the entire disclosure of which is incorporated herein by reference in its entirety.

### TECHNICAL FIELD

The present disclosure relates to a medical device, which is a heart harness, composed of a hollow structure filled with cells, and a treating method for cardiac insufficiency by use of the device.

### BACKGROUND DISCUSSION

As a method for culturing a large quantity of cells continuously and efficiently, there is known a culture method utilizing a hollow fiber membrane (refer to, for example, Ye et al., Biomaterials 27 (2006) 1955-1962 and Japan Patent Application Publication Nos. 2010-523118 and 2009-540865). In the culture utilizing the hollow fiber membrane, continuous reflux of a culture medium permits constant supply of a fresh medium. In addition, proteins, waste matter and the like produced by cells are discharged to the exterior of the hollow fiber membrane, whereby damage to the cells by impurities and the like can be prevented. Hollow fiber cell culture modules using such a culture method are commercially available. The cells thus cultured by use of the hollow fiber membrane are usually recovered by dissecting from the hollow fiber membrane, after the culture is completed.

In addition, in Kim et al., J Control Release, 2005 Jan. 20; 102(1): 101-11, a device is described made from hollow fiber membrane and capable of being filled with cells was transplanted into the brain of a rat and the device was filled with PC12 cells, then expression of tyrosine hydroxylase was confirmed in the site adjacent to the site where the device was transplanted.

As a medical device to be used for heart diseases, there is known a heart corrective net (heart harness) to be mounted to the heart (refer to, for example, Japanese Patent Application Publication No. 2013-183994). This device, which is mounted to the outside of the heart, is for physically restraining cardiac hypertrophy in a patient suffering from cardiac insufficiency, so as to prevent worsening of the cardiac insufficiency. Therefore, this is essentially a medical device for use on a symptomatic therapy basis.

In recent years, for repair of injured tissues, various attempts to transplant cells have been made; for example, for repair of cardiac muscle tissues injured by an ischemic heart disease such as stenocardia and cardiac infarction, attempts to utilize fetal cardiac myocytes, skeletal myoblasts, mesenchymal stem cells, cardiac stem cells, embryonic stem (ES) cells, etc. have been made (refer to, for example, Haraguchi et al., Stem Cells Transl Med. 2012 February; 1(2): 136-41). During such attempts in recent years, it has come to be known that cytokines excreted by the transplanted cells are useful for regeneration of the tissues even more than the engraftment and differentiations of the transplanted cells themselves into tissues (refer to, for example, Nakagami et al., J Atheroscler Thromb. 2006 April; 13(2): 77-81). However, a medical device capable of continuous supply of a cytokine to a heart disease site has not been known so far, thus supply of a cytokine by transplantation of a sheet-shaped cell culture is being conducted at present.

### SUMMARY

A new medical device is disclosed that can be used for cardiac insufficiency on a causal therapy basis.

In consideration of the above-mentioned circumstances, a medical device is disclosed, which is capable of continuous supply of a cytokine to a heart disease site. In accordance with an exemplary embodiment, a device is disclose, which can be mounted onto a heart in such a manner that the device will be capable of continuous release of a cytokine.

In an aspect, the present disclosure relates to the following:

A medical device is disclosed characterized in being a heart harness composed of a porous hollow structure.

In the medical device as above, the hollow structure is for being filled with cells.

In the medical device as above, the hollow structure is hollow fibers.

In the medical device as above, the hollow fibers are filled with cells by hollow fiber cell culture.

In the medical device as above, the cells are cytokine-producing cells.

In the medical device as above, the cells are skeletal myoblasts.

The medical device as above has a needle structure on an inner side of the device.

In the medical device as above, the needle structure is a hollow needle having a length of 5 mm to 10 mm.

In the medical device as above, the hollow structure is composed of an elastic (expandable and contractible) high-molecular compound.

In the medical device as above, the high-molecular compound is a biocompatible polymer.

In another aspect, the present disclosure also relates to the following:

A method of treating a heart disease, comprising applying the medical device as above to a heart of a subject in need thereof.

The method as above, wherein the medical device is composed of the hollow structure filled with cells.

The method as above, wherein the hollow structure of the medical device is a hollow fiber.

The method as above, wherein the hollow structure of the medical device is a hollow fiber and is filled with cells by hollow fiber cell culture.

The method as above, wherein the cells are cytokine-producing cells.

The method as above, wherein the cells are skeletal myoblasts.

The method as above, wherein the medical device has a needle structure on an inner side of the device.

The method as above, wherein the needle structure is a hollow needle having a length of 5 mm to 10 mm.

The method as above, wherein the medical device is composed of a hollow structure composed of an elastic high molecular compound.

The method as above, wherein the high molecular compound is a biocompatible polymer.

The device disclosed herein, when used as a harness for a heart, is able to treat cardiac hypertrophy (cardiac remodeling) on a symptomatic therapy basis. Moreover, the device, through the use of the porous hollow structure filled with cells that express a cytokine and the like as a member thereof, is able to supply the cytokine continuously while kept mounted onto the heart. As a result, the device is able to urge regeneration of cardiac tissues. Furthermore, since the device does not involve direct transplantation of cells, the required cells can be used without worrying, for example, about immunological rejection or the like.

### BRIEF DESCRIPTION OF THE DRAWINGS

FIG. 1 is a schematic view showing the manner in which a net-shaped heart harness, which is an embodiment of a medical device disclosed herein, is applied to a heart; and

FIG. 2 is a schematic view showing the manner in which a device of the embodiment having needle structures on an inner side is applied to a heart.

### DETAILED DESCRIPTION OF THE PREFERRED EMBODIMENTS

All the technical terms and scientific terms used herein have the same meanings as usually understood by one skilled in the art, unless otherwise defined herein. All the patents, applications, published applications, and other publications are incorporated herein by reference in their entirety. In addition, when a contradiction occurs between the publication referred to herein and the description of the present specification, the description of the present specification is given priority over the publication.

An aspect of the present disclosure relates to a medical device that is a heart harness composed of a porous hollow structure.

The term “heart harness” used herein refers to a device which is mounted on a patient's heart to prevent cardiac insufficiency from worsening. The heart has a property of changing its structure, upon exertion of a load thereon, to cope with the change in situation, and this phenomenon is called remodeling. It is known that in a patient suffering from cardiac insufficiency, the function of the cardiac muscles as a pump is lowered, and remodeling occurs in order to compensate for the lowering in the function, resulting in hypertrophy of the cardiac muscles, particularly, the cardiac wall for sending blood out to the whole body. Since the hypertrophy shortens the life of the heart, it is important to minimize the cardiac hypertrophy. A heart harness is a device for treating cardiac insufficiency on a symptomatic therapy basis (for preventing worsening of the cardiac insufficiency) by physically suppressing the progress of the cardiac hypertrophy due to remodeling.

The term “porous” used herein means the presence of a multiplicity of pores (holes) sized in such a manner that molecules smaller than a certain size can pass therethrough but particles of a certain size or greater cannot pass therethrough. Specifically, for example, the “porous” means the presence of pores (holes) through which cells themselves cannot pass but proteins produced by the cells can pass, the pores being typically pores having a pore diameter of approximately 0.2 μm. A membrane having a porous structure is also called “porous membrane” or “semi-permeable membrane”.

The medical device disclosed herein is characterized by being a heart harness composed of a porous hollow structure. Thus, an effective ingredient with which the hollow structure is filled or which is produced inside the hollow structure can be discharged to the exterior of the hollow structure, thus provided to the heart, through the porous membrane constituting the device disclosed herein. As a result, it is possible not only to produce a symptomatic therapeutic effect as the heart harness but also to produce a radical therapeutic effect by supply of cytokine.

The shape of the device disclosed herein may be any shape that is known as a shape of a heart harness. For example, the device may be net-shape, spring-shape, or belt-shape, and, for example, is preferably net-shape. In accordance with an exemplary embodiment, the device has a shape in which hollow fibers are knitted into a net shape, for example.

In an embodiment of the present disclosure, the hollow structure, for example hollow fibers, is filled with cells. In other words, the hollow structure constituting the medical device disclosed herein is typically for being filled with cells. The medical device is used in the state where the hollow structure is filled with cells, whereby a biologically active agent produced by the cells can be discharged to the exterior of the hollow structure through the porous membrane and be provided for the heart. Accordingly, in a preferred embodiment, the present disclosure provides a heart harness composed of a porous hollow structure filled with cells.

As the method for filling the hollow structure with cells, any method that is known in this technical field can be used. Non-limitative examples of the method can include a method in which cells are let flow into the hollow structure to fill the structure, and a method in which cells are cultured in the hollow structure until becoming confluent.

While the cells with which to fill the hollow structure may be any cells, the cells that produce a cytokine showing a suitable action on the heart may be preferable. Non-limitative examples of “the cytokine showing a suitable action on the heart” can include hepatocyte growth factor (HGF), vascular endothelial growth factor (VEGF), stromal cell-derived factor-1 (SDF-1), and leukemia inhibitory factor (LIF). Non-limitative examples of the cells that produce such a cytokine include skeletal myoblasts, mesenchymal stem cells, cardiac stem cells, bone marrow stem cells, and fibroblasts. In accordance with an exemplary embodiment, these cells may be those which are obtained by differentiation from stem cells. Non-limitative examples of such stem cells can include embryonic stem cells, mesenchymal stem cells, and induced pluripotent stem (iPS) cells. In addition, cells having undergone transformation such as to express a cytokine showing a suitable action on the heart may also be used.

The device disclosed herein is composed of a structure having pores (holes) of such a size that cell bodies themselves cannot pass therethrough but proteins can pass therethrough, for example, a porous membrane. Therefore, the cells in the structure are less liable to be subjected to immunological rejection at the object of transplantation, and, accordingly, the cells with which to fill the device of the present disclosure may be autologous cells or may be allologous cells. From the viewpoint of safety, however, the cells are preferably autologous cells.

In a preferred embodiment of the device of the present disclosure, the hollow structure is hollow fibers. A cell culture system using hollow fibers is known in this technical field. Accordingly, a method of filling hollow fibers with cells is also known, and, in the case of filling the hollow fibers in this embodiment with cells, any method that is known in the technical field may be used. In a preferred embodiment of the present disclosure, the filling with cells is conducted by hollow fiber culturing.

In such an embodiment, the hollow fibers are knitted in a net-shape, and, in the knitted state, the fibers are subjected to cell culture or filled with cells. From the viewpoint of ease of operation, the hollow fibers in the knitted state are subjected to cell culture. The hollow fiber structure in such an embodiment can be produced in such a form as to be usable in a hollow fiber cell culture module that is commercially available.

After the cell culture or the filling with cells, the hollow fiber device of the present disclosure can be developed into a net shape and applied to the heart of the subject. As a result, a cytokine produced from the cells can be continuously supplied to the heart, while physically restraining cardiac hypertrophy due to remodeling of the heart by the function of the device as a heart harness.

As the material constituting the hollow structure herein, any material that can form a porous membrane may be used. Non-limitative examples of such material can include cellulose, cellulose esters, polysulfones, polyether sulfones, polyacrylonitrile, polyvinyl alcohol, polystyrene, and polymethyl methacrylate.

Since the medical device disclosed herein is a heart harness for restraining excessive dilatation of the heart, its ability to restrain cardiac hypertrophy is lowered if its constraining force on the heart to which it is applied is too weak. If the constraining force is too strong, on the other hand, it may lead to diastolic disorder. Therefore, the material of the medical device of the present disclosure is preferably an elastic (expandable and contractible) material, particularly an elastic high-molecular compound. Examples of the elastic high-molecular compound that can be used for the medical device disclosed herein can include, e.g., polylactic acid-cholesterol conjugate (P_{DL}LA-cholesterl). In addition, since the medical device of the present disclosure is to be applied to a living body, the material thereof is preferably a biocompatible polymer. Thus, in a more preferable embodiment, the device of the present disclosure is produced by use of an elastic and biocompatible polymer. Examples of such a polymer typically can include, e.g., polylactic acid-cholesterol conjugate (P_{DL}LA-cholesterl).

In a preferred embodiment of the device of the present disclosure, an inner wall of the hollow structure has been treated to have cell adhesiveness. The treatment for imparting cell adhesiveness may be any one that is known in this technical field, and examples of the treatment for imparting cell adhesiveness can include a treatment for imparting hydrophilicity, and cell adhesive coating. Thereby, cells can be cultured in an adhered state with the inner wall of the hollow structure as a scaffold. Accordingly, it is also possible to achieve such adhesion that the cells form a monolayer sheet-shaped structure on the inner wall of the hollow structure.

In addition to the cell adhesiveness imparting treatment, an adhesion promoting treatment may further be conducted. As the adhesion promoting treatment, any adhesion promoting treatment that is known in this technical field can be used. Non-limitative examples of such an adhesion promoting treatment can include a plasma treatment, a corona discharge treatment, an ultraviolet (UV) irradiation treatment, and coating with a cell-adhesive material. For example, such an adhesion promoting treatment may be performed partially. For example, in the case where the device is applied to a heart as a heart harness, the adhesion promoting treatment can be applied to the inner wall of the hollow structure at a portion located at that part of the subject at which infarction is occurring. Thereby, in the case where the device is applied as a harness, more cells can be localized at that portion located at the infarcted part, so that the therapeutic effect by cytokine can be enhanced. Thus, by adjusting the part to which the adhesion promoting treatment is applied and the degree of the treatment, the localization of cells in the hollow structure can be controlled.

In a preferred embodiment, the device of the present disclosure has a needle structure or structures on an inner side of the device. The expression “inner side of the device” means the heart side of the device. With the needle structures provided on the inner side of the device, the device can be fixed to the heart. Further, where the needles are hollow needles, the cytokine produced inside the hollow structure can be delivered into cardiac muscles through the needle portions. In such an embodiment, it can be preferable that at least distal portions of the needles penetrate the pericardium to reach at least the pericardial cavity, preferably the cardiac muscles. Therefore, it is preferable that the length of the needles is greater than the thickness of the pericardium. In accordance with an exemplary embodiment, since the thickness of the pericardium can be, for example, approximately 3 mm, the length of the needles can be, for example, approximately 3 mm to 10 mm, preferably approximately 5 mm to 10 mm.

In another exemplary embodiment of the present disclosure, a capillary vessel-like sheet structure connected to the hollow structure is provided on the inner side of the device. The capillary vessel-like sheet structure can be embedded in the pericardium, whereby the cytokine produced inside the hollow structure can be suitably delivered into the pericardium.

In a further exemplary embodiment of the present disclosure, the device has a filling pod connected to the hollow structure. The filling pod is to be used in such a manner that a needle or the like for filling is made to pierce the pod and cells or a cytokine solution is injected, whereby the hollow structure can be refilled with living cells or the cytokine solution.

The device disclosed herein can be preferably used for treatment of mainly cardiac insufficiency, particularly ischemic or chronic myocardial infarction or dilated cardiomyopathy. Therefore, in an exemplary aspect, the present disclosure provides a method of treating a heart disease by use of the device disclosed herein. The device of the present disclosure is able to help prevent the progress of a heart disease by inhibiting cardiac remodeling on a symptomatic therapy basis through functioning as a heart harness, and to treat the heart disease on a causal therapy basis by functioning as a cytokine-supplying device.

The heart disease that can be treated by the treating method disclosed herein is typically cardiac insufficiency, as mentioned above, and examples thereof include ischemic or chronic myocardial infarction, cardiac trauma, and cardiomyopathy inclusive of dilated cardiomyopathy.

The treating method of the present disclosure can include the following steps:

(a) providing a heart harness composed of a porous hollow structure;

(b) optionally introducing to a hollow structure portion of the heart harness a supply source of a cytokine showing a suitable action on the heart; and

(c) applying the heart harness to the heart of the subject in need of the treatment.

In the step (a), the heart harness composed of the porous hollow structure is provided. The details of the heart harness thus provided are as described above with reference to the device of the present disclosure. The porous hollow structure can be hollow fibers, but is not limited to the hollow fibers. For example, hollow fibers may be knitted into a net shape, or hollow structures such as hollow fibers may be connected into a mesh-like shape to form a hollow structure net, which may be formed into a bag shape or a belt shape, whereby the heart harness composed of a porous hollow structure can be provided.

To the lumen of the hollow structure, there can be applied an arbitrary treatment useful in the subsequent step or steps and/or in the use of the device of the present disclosure. For example, in the case where cells are to be introduced into the hollow structure, in order that more cells are liable to be accumulated in that portion of the hollow structure which corresponds to the affected part when the device is used as the harness, an adhesion promoting treatment may be applied predominantly to the inner wall of the hollow structure that is to be brought into contact with the diseased site.

In the step (b), a supply source of a cytokine showing a suitable action on the heart is introduced into the hollow structure of the heart harness provided in the step (a). Since the device disclosed herein itself functions as a heart harness, the step (b) may be or may not be conducted. Whether or not the step (b) is to be conducted can be determined depending on the kind of the disease to be treated by application of the heart harness, the severity of the disease, etc.

Examples of the supply source of a cytokine include a cytokine-containing solution, and cells that produce a cytokine. In the case of introducing cells (or filling with cells), the cells are not particularly limited so long as they express a cytokine showing a suitable action on the heart. The cells may naturally express such a cytokine, or may have undergone transformation such as to express the cytokine. Examples of the cells that naturally express a cytokine are as above-mentioned, and are particularly preferably skeletal myoblasts.

Filling of the hollow structure with the cells can be achieved by any technique that is known in this technical field. Examples of such a technique can include injecting a cell suspension into the hollow structure, and introducing the cells into the hollow structure and thereafter culturing the cells until becoming confluent by hollow fiber cell culture using, for example, a hollow fiber cell culture module.

In the step (c), the heart harness is applied to the heart of the subject in need of the treatment. The subject in need of the treatment can be a subject having a heart disease. The heart harness, after filling it with the supply source of a cytokine, for example, after introducing cytokine-producing cells such as skeletal myoblasts into the hollow fibers and thereafter culturing the cells until becoming confluent by using a hollow fiber cell culture module, the heart harness may be detached from the module, expanded, and applied to the heart. The application of the heart harness to the heart can be carried out by a surgical operation.

Since the method disclosed herein can exert a considerable burden on the subject at the time of application to the subject, it is preferable that the number of times of application to the subject is as few as possible. In addition, the function of the device as the heart harness is exhibited lastingly. On the other hand, the cytokine-supplying function of the device may be lowered with the lapse of time, due to depletion of the cytokine introduced into the device and/or death of the cells filled in the device. Therefore, in an embodiment of the present disclosure, the device may be refilled with the cytokine supply source in a low-invasive manner. The refilling can be achieved, for example, by a method in which the cytokine supply source is injected into a filling pod connected to the hollow structure. The refilling with the cytokine supply source may be carried out multiple times.

The present disclosure will be described more in detail below referring to specific embodiments of the disclosure. The following specific embodiments are the preferable embodiments of the present disclosure, but are not to be construed as restrictive.

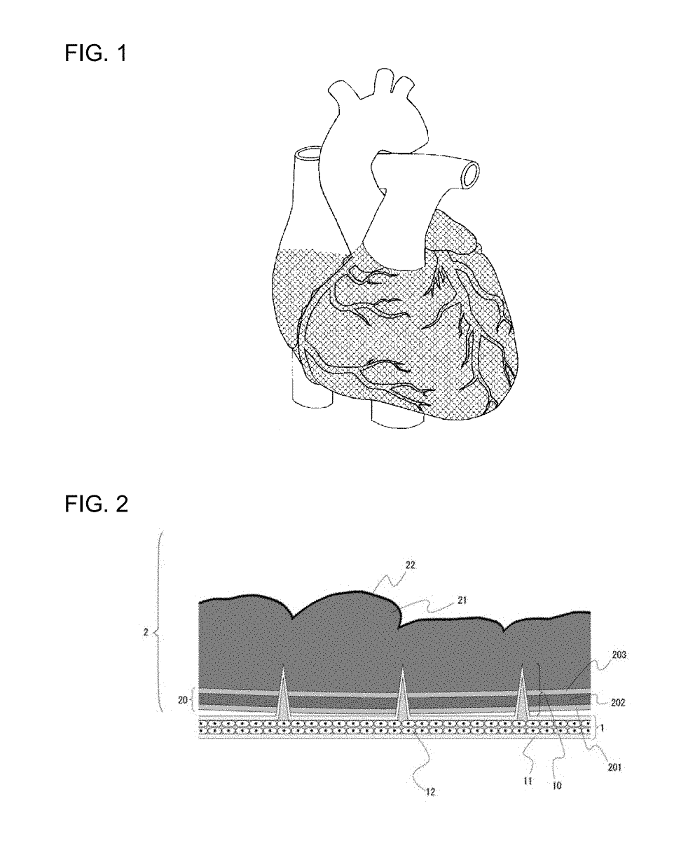

FIG. 1 is a schematic view showing the manner in which a medical device according to an embodiment of the present disclosure that is composed of hollow fibers is applied to a heart. In this embodiment, the device of the present disclosure is in a bag-like net shape, but it may be in a belt-like net shape so long as it can physically cover an infarcted part. In addition, by changing the manner in which the hollow fibers are knitted, the contracting (shrinking) force of the harness can be changed. Specifically, by knitting the hollow fibers densely, a strong contracting force can be obtained.

In the device in the aforesaid embodiment, after the hollow fibers are knitted into a net shape, the hollow fiber net is attached to a commercialized hollow fiber cell culture module, and skeletal myoblasts may be cultured in the module, whereby the hollow structure of the device of the present disclosure can be filled with the skeletal myoblasts. Thereafter, the hollow fiber net can be detached from the module and be applied, as it is, to a heart.

When the device in the present embodiment filled with the skeletal myoblasts is applied to the heart, the cytokine such as HGF, VEGF, or SDF-1 excreted from the skeletal myoblasts at the heart is delivered to the heart, to induce angiogenesis in the infarcted part, whereby cardiac insufficiency can be treated.

FIG. 2 is a schematic view showing the manner in which a medical device 1 of the preset disclosure in an embodiment of having needle structures 10 is applied to a heart 2 (consisted of pericardium 20 cardiac muscle 21 and endocardium 22). In this embodiment, the needles 10 have such a length that the distal of the needles penetrate the pericardium 20 (consisted of cardiac sac 201, pericardial cavity 202 and epicardium 203) to reach the cardiac muscles 21. Specifically, the needle length is preferably, for example, not less than 3 mm, more preferably approximately 3 mm to 10 mm, and still more preferably approximately 5 mm to 10 mm. The medical device 1 disclosed herein, or the heart harness, is produced by knitting hollow fibers 11, and the hollow fibers 11 are filled with skeletal myoblasts 12 by a method in which a net knitted from the hollow fibers 11 is subjected to hollow fiber module culture. When this heart harness 1 is applied to a heart 2, the needles 10 penetrate the pericardium 20 to reach cardiac muscles 21 and a cytokine is supplied via the needle portions, so that the cytokine useful for regeneration of the cardiac muscles 21 can be sufficiently supplied to the cardiac muscles.

According to the present disclosure, a medical device capable of treating a heart disease, particularly cardiac insufficiency, on a causal therapy basis is provided. The device disclosed herein is an epoch-making device in that it is possible, while using a heart harness which could conventionally been used only on a symptomatic therapy basis, to achieve even regeneration of cardiac muscles in an infarcted part. In addition, the device of the present disclosure helps enable a biologically active agent produced by the cells filling the device to be delivered to the heart sustainedly and in a suitably manner, so that a further effect in cardiac muscle regenerative therapy can thereby be expected.

The detailed description above describes a medical device that can be used for cardiac insufficiency on a causal therapy basis. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
a heart harness composed of a porous hollow structure.

2. The medical device according to claim 1, wherein the hollow structure is filled with cells.

3. The medical device according to claim 1, wherein the hollow structure is a hollow fiber.

4. The medical device according to claim 2, wherein the hollow structure is a hollow fiber, and the hollow structure is filled with cells by hollow fiber cell culture.

5. The medical device according to claim 2, wherein the cells are cytokine-producing cells.

6. The medical device according to claim 2, wherein the cells are skeletal myoblasts.

7. The medical device according to claim 1, further comprising:
a needle structure on an inner side of the medical device.

8. The medical device according to claim 7, wherein the needle structure is a hollow needle having a length of 5 mm to 10 mm.

9. The medical device according to claim 1, wherein the hollow structure is composed of an elastic high molecular compound.

10. The medical device according to claim 9, wherein the elastic high molecular compound is a biocompatible polymer.

11. A method of treating a heart disease, comprising:
applying the medical device according to claim 1 to a heart of a subject.

12. The method according to claim 11, wherein the medical device is composed of the hollow structure filled with cells.

13. The method according to claim 11, wherein the hollow structure of the medical device is a hollow fiber.

14. The method according to claim 12, wherein the hollow structure of the medical device is a hollow fiber and the hollow structure is filled with cells by hollow fiber cell culture.

15. The method according to claim 12, wherein the cells are cytokine-producing cells.

16. The method according to claim 12, wherein the cells are skeletal myoblasts.

17. The method according to claim 11, wherein the medical device has a needle structure on an inner side of the device.

18. The method according to claim 17, wherein the needle structure is a hollow needle having a length of 5 mm to 10 mm.

19. The method according to claim 11, wherein the medical device is composed of a hollow structure composed of an elastic high molecular compound.

20. The method according to claim 19, wherein the high molecular compound is a biocompatible polymer.
